(19) United States
(12) Patent Application Publication
(10) Pub. No.: US 2021/0244700 A1
(43) Pub. Date: Aug. 12, 2021

(54) COMPOSITIONS AND METHODS FOR PREVENTING AND/OR REDUCING ISCHEMIA AFTER SURGICAL INCISIONS

(71) Applicant: Duke University, Durham, NC (US)

(72) Inventors: David BROWN, Durham, NC (US); Scott HOLLENBECK, Durham, NC (US)

(21) Appl. No.: 17/055,570

(22) PCT Filed: May 15, 2019
(86) PCT No.: PCT/US2019/032438

§ 371 Date: Nov. 14, 2020

Related U.S. Application Data
(60) Provisional application No. 62/671,697, filed on May 15, 2018.

(51) Int. Cl. A61K 31/225; A61K 9/00; A61P 17/02; A61K 9/06

(52) U.S. Cl. CPC A61K 31/225; A61K 9/06; A61P 17/02; A61K 9/0014

(57) ABSTRACT

The present invention relates to methods of reducing ischemic damage to a surgical incision in a tissue of subject, enhancing tissue viability and vascularity following an ischemic event, and preconditioning tissue to resist an ischemic insult, which comprises contacting the relevant tissue topically with an effective dose of a HIF-1 potentiating agent, thereby reducing ischemic damage to a surgical incision, enhancing tissue viability and vascularity following an ischemic event, and preconditioning tissue to resist an ischemic insult.

### FIELD OF THE INVENTION

The present invention relates to methods of reducing ischemic damage to a surgical incision in a tissue of subject, enhancing tissue viability and vascularity following an ischemic event, and preconditioning tissue to resist an ischemic insult, which comprises contacting the relevant tissue topically with an effective dose of a HIF-1α potentiating agent, thereby reducing ischemic damage to a surgical incision, enhancing tissue viability and vascularity following an ischemic event, and preconditioning tissue to resist an ischemic insult.

### BACKGROUND

Despite surgical advances and continued research into the mechanisms governing necrosis, tissue ischemia and flap necrosis remain critical complications leading to morbidity and excess healthcare expenditures. Given that necrosis of the distal skin flap is thought to stem from insufficient arterial blood supply or lack of venous outflow, the “delay” phenomenon, involving invoking an ischemic insult to stimulate vascular rerouting and angiogenesis toward the distal flap, is an actively-investigated adjunct to flap surgery. Surgical delay procedures are currently the most reliable means of improving flap survival through ischemic preconditioning, but are limited by invasiveness and requirement for a two-stage operation. Thus, achieving a delay phenomenon through pharmacologic tissue preconditioning is a highly sought-after solution to distal flap ischemia. Based on the neo-vascularization integral to the delay phenomenon, research into tissue preconditioning thus far has largely focused on vascular remodeling agents including vasodilators, VEGF delivery vehicles, minoxidil, and octreotide. However, while these strategies have achieved marginal improvements in flap viability, they lack clinical application and utility.

Inherent to the idea of neo-angiogenesis driving the delay phenomenon is that metabolic adaptation to ischemia acts as the primary stimulus for vascular changes. Accordingly, preconditioning agents manipulating pathways involving metabolic adaptation to hypoxia may represent a more efficacious approach to resist ischemic insult. The hypoxia inducible factor (HIF) pathway has recently gained attention as a key mediator of tissue ischemia under hypoxic conditions. HIF-1α, a DNA-binding transcription factor, serves a protective role against ischemia by inducing transcription of genes including vascular endothelial growth factor (VEGF) and erythropoietin (EPO). However, HIF-1α becomes marked for proteasomal degradation by prolyl hydroxylase (PHD) under hypoxic conditions. Thus, PHD inhibition has been shown to protect against tissue ischemia through promotion of HIF-1alpha-induced transcription and neo-angiogenesis. PHD inhibitors have shown considerable promise throughout phase 2 and 3 clinical trials as novel agents to treat chronic kidney disease-induced anemia. Furthermore, PHD inhibitors have also demonstrated utility in pre-clinical trials of reduction of organ rejection post-transplant, treatment of atherosclerosis, and mitigation of parenchymal injury following ischemic stroke.

There is a need for compositions and methods that promote PHD inhibition to improve viability of an ischemic skin flap.

### BRIEF SUMMARY OF THE INVENTION

The disclosure provides a method of reducing ischemic damage to a surgical incision in a tissue of subject, which comprises contacting said surgical incision topically with an effective dose of a HIF-1α potentiating agent, thereby reducing ischemic damage to the surgical incision.

The disclosure further provides a method of enhancing tissue viability and vascularity following an ischemic insult in a subject, which comprises contacting said tissue topically with an effective dose of a HIF-1α potentiating agent, thereby enhancing tissue viability and vascularity following the ischemic insult.

The disclosure also provides a method for preconditioning tissue to resist an ischemic insult, which comprises contacting said tissue topically with an effective dose of a HIF-1α potentiating agent prior to the ischemic insult.

Also provided is the use of a lotion or gel comprising a HIF-1α potentiating agent for reducing ischemic damage to a surgical incision in a tissue of subject, for enhancing tissue viability and vascularity following an ischemic insult in a subject. and for preconditioning tissue to resist an ischemic insult.

### BRIEF DESCRIPTION OF THE DRAWING(S)

FIG. 1 is a schematic diagram showing flap design and experimental time course. Fourteen-day experimental time course, with image pattern on day 1 depicting location of daily topical treatment in the outline of the proposed skin flap, and right flap illustrating representative distances from which immunohistochemical samples were collected (TUNEL, terminal deoxynucleotidyl transferase-mediated dUDP end labeling; IP, intraperitoneal).

FIG. 2 includes a series of images of skin flaps one day after surgery in animals treated with both intraperitoneal and topical treatment (24 mg/kg/day dimethyloxalylglycine—top images) versus controls (bottom images).

FIG. 3A includes images of flap necrosis 7 days after surgery with dimethyloxalylglycine dose increasing from controls (left) to 48 mg/kg/day (right). FIG. 3B is a graph showing mean percentage of distal necrosis versus controls 3 days after surgery (**p<0.01; ***p<0.001). FIG. 3C is a graph showing mean percentage of distal necrosis versus controls 7 days after surgery (**p<0.01; ***p<0.001).

FIG. 4A includes fluorescent images of skin flaps in control and dimethyloxalylglycine-treated animals after sodium fluorescein injection. Gray regions represent areas with critical tissue perfusion. FIG. 4B is a graph showing percentage of unperfused tissue in control versus dimethyloxalylglycine-treated animals. FIG. 4C is a response curve demonstrating increase in tissue perfusion with dimethyloxalylglycine dose delivered.

FIGS. 5A and 5B are graphs showing necrosis and tissue perfusion on postsurgical day 7 in animals treated with topical dimethyloxalylglycine or intraperitoneal (IP) dimethyloxalylglycine alone versus controls. FIG. 5A shows mean percentage of distal necrosis versus controls 7 days after surgery (**p<0.01; ***p<0.001). FIG. 5B shows percentage of unperfused tissue in controls versus animals treated with topical or intraperitoneal dimethyloxalylglycine (***p<0.001).

FIG. 6A includes representative immunohistochemical images of HIF-1α staining in dimethyloxalylglycine-treated (left) versus control (right) skin flaps, with tissue sections harvested 4 cm from the proximal flap adjacent to the pedicle. Scale bars=50 μm. Epidermal areas positive for HIF-1α are identified by red chromogen, with sections showing increased numbers of HIF-1α-stained nuclei in the epidermis of dimethyloxalylglycine-treated flaps. FIG. 6B is a graph showing the number of HIF-1α-stained nuclei in dimethyloxalylglycine-treated versus control flaps (***p<0.001) (hpf, high-power field).

FIGS. 7A and B include histological images showing is shows neovascularization from dimethyloxalylglycine (DMOG) treatment. Scale bars=50 μm. FIG. 7A shows CD31-stained tissue sections from skin flaps harvested 6 cm from the proximal flap adjacent to the pedicle in treated (top) versus control animals (center), with increased numbers of CD31-stained vessels in dimethyloxalylglycine-treated rats. FIG. 7B includes images of hematoxylin and eosin-stained tissue sections from skin flaps harvested 6 cm from the proximal flap adjacent to the pedicle in treated (top) versus control animals (center), with enhanced neovascularization seen in dimethyloxalylglycine-treated rats. FIG. 7C is a graph showing the number of CD31+(brown) vessels in treated versus control animals, reported as number of vessels per high-power field (hpf) at 20× magnification (**p<0.01). FIG. 7D is a graph showing tissue concentration of VEGF (in picograms per milliliter) measured with enzyme-linked immunosorbent assay 6 cm from the proximal flap adjacent to the pedicle in treated versus untreated animals (**p<0.01).

FIGS. 8A and 8B show effects of dimethyloxalylglycine (DMOG) treatment on apoptosis. Scale bars=50 μm. FIG. 8A includes images of nonnecrotic sections of skin flaps taken from an equal distance (4 cm from the proximal flap) that were stained with terminal deoxynucleotidyl transferase-mediated dUDP end-labeling, with brown apoptotic bodies in the epidermal and dermal layers demonstrated at 10× magnification (arrows). FIG. 8B is a graph showing the number of apoptotic cells per high-power field (hpf) at 20× magnification in treatment and control animals (*p<0.05).

FIGS. 9A and 9B are graphs which show physiologic parameters in treated versus untreated rats. FIG. 9A shows complete blood counts after 14 total days of treatment in treated versus untreated animals. FIG. 9B shows weights taken on experiment day 1 and day 14 for control animals and all doses of treated animals.

FIG. 10 is a schematic diagram showing the simplified HIF pathway. Hypoxic conditions or prolyl hydroxylase (PHD) inhibitors (DMOG) enable HIF-1α binding to hypoxia-response elements, leading to increased transcription of proangiogenic and erythropoietic (EPO) genes (VHL, von Hippel-Lindau protein; FGF, fibroblast growth factor).

### DETAILED DESCRIPTION OF THE INVENTION

The present disclosure is predicated, at least in part, on the discovery that topical and systemic targeting of the HIF-1 pathway reduces necrosis in a rat ischemic skin flap model and may be a promising therapeutic approach to improve flap resistance to ischemia following surgical insult.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

Unless otherwise defined, all technical terms used herein have the same meaning as commonly understood by one of ordinary skill in the art to which this disclosure belongs.

“About” is used to provide flexibility to a numerical range endpoint by providing that a given value may be “slightly above” or “slightly below” the endpoint without affecting the desired result.

Recitation of ranges of values herein are merely intended to serve as a shorthand method of referring individually to each separate value falling within the range, unless otherwise-indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The terms “effective amount” or “therapeutically effective amount” refers to an amount sufficient to effect beneficial or desirable biological and/or clinical results.

As used herein, the terms “subject” and “patient” are used interchangeably herein and refer to both human and nonhuman animals. The term “nonhuman animals” includes all vertebrates, e.g., mammals and non-mammals, such as nonhuman primates, sheep, dogs, cats, horses, cows, chickens, amphibians, reptiles, and the like.

As used herein, the terms “surgical wound,” “surgical incision,” and “surgical insult” are used interchangeably and refer to a cut/wound made through the skin and soft tissue of a subject to facilitate an operation or procedure. A surgical wound may comprise one wound or many wounds and are dependent on the type of surgery being performed. As used herein, the term “skin flap” refers to healthy skin and tissue that is partly detached and (sometimes) moved to cover a nearby surgical wound/incision. The skin flap may contain skin, fat, and/or muscle. Often, the skin flap is still attached to its original site at one endo and remains connected to a blood vessel.

The term “ischemia” refers to a restriction in blood supply to a tissue or organ, which causes a shortage of oxygen. An “ischemic insult” is a cut or wound made through the skin or soft tissue of a subject that induces ischemia.

The term “pharmaceutically acceptable” as used herein refers to a compound or combination of compounds that will not impair the physiology of the recipient human or animal to the extent that the viability of the recipient is compromised. Preferably, the administered compound or combination of compounds will elicit, at most, a temporary detrimental effect on the health of the recipient human or animal.

The term “carrier” as used herein refers to any pharmaceutically acceptable solvent of agents that will allow a therapeutic composition to be administered directly to a wound of the skin. The carrier will also allow a composition to be applied to a medical dressing for application to such a wound. A “carrier” as used herein, therefore, refers to such solvent as, but not limited to, water, saline, physiological saline, ointments, creams, oil-water emulsions, gels, or any other solvent or combination of solvents and compounds known to one of skill in the art that is pharmaceutically and physiologically acceptable to the recipient human or animal.

The present disclosure provides, in part, compositions and methods for reducing ischemia after a surgical incision. It has been found that the hypoxia-inducible factor (HIF) pathway is central to tissue adaptation to ischemic conditions, and that activation of the HIF pathway is regulated by prolyl hydroxylase (PHD). As described herein, addition of a HIF-1α potentiation agent can reduce ischemic damage to surgical wounds/incisions and surgically-induced skin flaps, thereby significantly enhancing tissue viability and vascularity.

In this regard, the disclosure provides a method of improving post-operative skin flap viability in an individual, the method comprising, consisting of, or consisting essentially of contacting said skin flap topically with an effective dose of a HIF-1α potentiating agent. In other embodiments, the present disclosure provides a method of reducing ischemic damage to a surgical incision in tissue of a subject, the method comprising, consisting of, or consisting essentially of contacting said surgical incision topically with an effective dose of a HIF-1α potentiating agent. The disclosure also provides a method of enhancing tissue viability and vascularity following an ischemic insult in a subject, the method comprising, consisting of, or consisting essentially of contacting said tissue topically with an effective dose of a HIF-1α potentiating agent. In certain embodiments, the HIF-1α potentiating agent transdermally penetrates the skin flap.

In another embodiment, the disclosure provides a method for preconditioning tissue to resist an ischemic insult, the method comprising, consisting of, or consisting essentially of contacting said tissue topically with an effective dose of a HIF-1α potentiating agent prior to the ischemic insult. In some embodiments, the HIF-1α potentiating agent may be administered 1-10 hours (e.g., 1, 2, 3, 4, 5, 6, 7, 8, 9, or 10 hours) prior to the ischemic insult. In other embodiments, the HIF-1α potentiating agent may be administered 1-7 days (e.g., 1, 2, 3, 4, 5, 6, or 7 days) prior to the ischemic insult. In other embodiments, the HIF-1α potentiating agent may be administered after an ischemic insult. For example, the HIF-1α potentiating agent may be administered 1-7 days (e.g., 1, 2, 3, 4, 5, 6, or 7 days) post ischemic insult. In some embodiments, the ischemic insult is a surgical incision.

The disclosure further provides the use of a lotion or gel comprising a HIF-1α potentiating agent for reducing ischemic damage to a surgical incision in a tissue of subject, for enhancing tissue viability and vascularity following an ischemic insult in a subject, and/or for preconditioning tissue to resist an ischemic insult.

As used herein, the term “HIF-1” includes both a heterodimer complex and the subunits thereof, HIF-1α and HIF-1. The HIF 1 heterodimer consists of two helix-loop-helix proteins; these are termed HIF-1α, which is the oxygen-responsive component and HIF-1β. The latter is also known as the aryl hydrocarbon receptor nuclear translocator (ARNT). In certain embodiments, the term “HIF-1” refers to the human form of HIF-1α. HIF-1α and its functions are further described in, e.g., Lee et al., Exp Mol Med., 36(1): 1-12 (2004)).

HIF-1α potentiating agents include agents that increase the accumulation or stability of HIF-1α; directly provide HIF-1α activity; or increase expression of HIF-1. Such agents are known in the art, or may be identified through art-recognized screening methods. Suitable HIF-1 potentiating agents include, but are not limited to, cofactor-based inhibitors such as 2-oxoglutarate analogues, ascorbic acid analogues and iron chelators such as desferrioxamine (DFO), the hypoxia mimetic cobalt chloride (CoC12), and mimosine, 3-Hydroxy-4-oxo-1(4H)-pyridinealanine, or other factors that may mimic hypoxia. In some embodiments, the HIF-1α potentiating agent may include hydroxylase inhibitors, including deferiprone, 2,2′-dipyridyl, ciclopirox, dimethyloxalylglycine (DMOG), L-Mimosine (Mim), and 3-Hydroxy-1,2-dimethyl-4(1H)-Pyridone (OH-pyridone). Other HIF hydroxylase inhibitors include, e.g., oxoglutarates, heterocyclic carboxamides, phenanthrolines, hydroxamates, and heterocyclic carbonyl glycines (including, but not limited to, pyridine carboxamides, quinoline carboxamides, isoquinoline carboxamides, cinnoline carboxamides, beta-carboline carboxamides, including substituted quinoline-2-carboxamides and esters thereof; substituted isoquinoline-3-carboxamides, and N-substituted arylsulfonylamino hydroxamic acids). In some embodiments, the HIF-1α potentiating agent upregulates expression of HIF-1α. In other embodiments, the HIF-1α potentiating agent inhibits the activity of prolyl hydroxylase (PHD). For example, the HIF-1α potentiating agent may comprise dimethyloxalylglycine (DMOG).

Dimethyloxalylglycine is a prolyl hydroxylase inhibitor under investigation in various clinical applications (see, e.g., Yuan et al., BMC Biotechnol. 2014; 14:112; Marchbank et al., Lab Invest. 2011; 91:1684-1694; Poynter et al., Surgery 2011; 150:278-283; Dallatu et al., J Hypertens (Los Angel.) 2014; 3:184; and Duscher et al., Plast Reconstr Surg. 2017; 139:695e-706e). The hydroxylase activity of prolyl hydroxylase depends on the presence of oxygen, iron(II), and 2-oxoglutarate as cofactors. Dimethyloxalylglycine, a 2-oxoglutarate analogue, results in competitive inhibition of prolyl hydroxylase-2-oxoglutarate interaction leading to reduced prolyl hydroxylase activity and subsequent increased HIF-1α-induced transcription (Semenza, G. L., Cell 2012; 148:399-408). The neoangiogenic benefits of prolyl hydroxylase inhibitors such as dimethyloxalylglycine are well known in the art (Yuan et al., supra, Marchbank et al., supra, Poynter et al., supra; and Dallatu et al., supra).

In some embodiments, the HIF-1α potentiating agent or agents is formulated for dosing, typically embedded or dispersed in a polymer for extended release of the agent. An effective dose of HIF-1α potentiating agent(s) may be determined by the practitioner and depends on type of HIF-1α potentiating agent, the route of administration, and patient characteristics (age, weight, sex, etc.). In general, the HIF-1α potentiating agent may be present at a concentration of at least about 1%, about 2%, about 3%, about 4% about 5%, about 8%, about 12% and not more than about 20% as weight/weight percent of polymer.

In some embodiments, the total dose of HIF-1α potentiating agent provided in topical delivery system (e.g., a transdermal patch, lotion, or gel) will be at least about 1 mg, usually at least about 5 mg, and not more than about 1000 mg, usually not more than about 500 mg, or not more than about 200 mg, and may be from about 10 mg to about 200 mg, e.g. about 100 mg.

The HIF-1α potentiating agent may be present in composition (e.g., a “pharmaceutically acceptable” composition) that may be formulated as a patch, lotion, gel, etc., and may further comprise additional agents involved in surgical incision/wound healing, e.g. transdermal penetration enhancers, anti-microbial agents, and the like. In certain embodiments, the HIF-1α potentiating agent is provided as a lotion or gel.

In embodiments where the formulation comprises a lotion or a gel, the formulation may include a therapeutically acceptable vehicle to act as a dilutant, dispersant, or carrier, so as to facilitate its distribution and uptake when the composition is applied to the skin. Vehicles other than or in addition to water can include liquid or solid emollients, solvents, humectants, thickeners and powders.

The timing of for administration a therapeutic formulation of the present disclosure, e.g. a lotion, will vary for prophylaxis or treatment. The dosage of HIF-1α potentiating agent can determine the frequency of drug depletion in a lotion, gel, or transdermal patch.

The following examples further illustrate the invention but, of course, should not be construed as in any way limiting its scope.

### Example 1

This example demonstrates that perioperative treatment with a prolyl hydroxylase inhibitor reduces necrosis in a rat ischemic skin flap model.

Materials and Methods Animal Care and Skin Flap Surgery

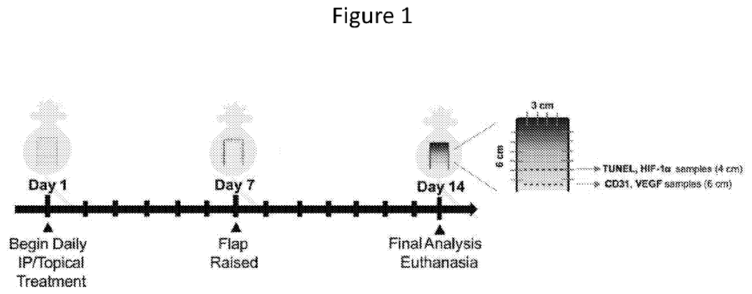

After institutional animal care and use committee approval, hairless male rats (8 weeks old; 250 g) were obtained from Charles River Laboratories (Wilmington, Mass.). Six experimental groups of rats (n=24) and three control groups (n=12) underwent skin flap surgery by surgeons blinded to the experimental treatment arm. First, to assess both systemic and topical administration together, four experimental groups (n=16) were administered dimethyloxalylglycine (Cayman Chemical, Ann Arbor, Mich.) both intraperitoneally and topically for a total dose of 6 mg/kg/day (n=4), 12 mg/kg/day (n=4), 24 mg/kg/day (n=4), or 48 mg/kg/day (n=4). Next, to assess topical versus intraperitoneal administration, two experimental groups were administered dimethyloxalylglycine at 48 mg/kg/day either topically (n=4) or intraperitoneally (n=4). Intraperitoneal administration consisted of dimethyloxalylglycine suspension in phosphate-buffered saline (pH 7.4) to a maximum dosage of 10 ml/kg. Topical delivery consisted of administration of dimethyloxalylglycine using dimethylsulfoxide as a delivery vehicle at a maximum dosage of 9 ml/kg undiluted solution. Topical application was performed in the outline of the proposed skin flap. To replicate a short therapeutic course before and after flap surgery, the study was designed to include a 14-day treatment period, with dimethyloxalylglycine administered 7 days before and 7 days after flap surgery (FIG. 1). Because prolyl hydroxylase inhibitors target the HIF pathway pre-transcriptionally, this design was chosen to allow time for downstream neovascularization.

Throughout the same 14-day treatment course, control groups were administered an equal volume of intraperitoneal phosphate-buffered saline (pH 7.4) and topical dimethylsulfoxide. A dorsal pedicle skin flap based on the McFarlane model measuring 3×6 cm was elevated in the areolar tissue plane deep to the panniculus carnosus layer on each rat on treatment day 7. Consistency in flap design was ensured using a model outline for size and shape and positioning according to bony landmarks. After flap elevation, the flap was sutured in place with 4-0 polypropylene. Surgeons were blinded intraoperatively to treatment arm with a random numbers scheme. Seven days after surgery, the animals were euthanized and histologic analysis performed in postmortem tissue specimens.

Skin Flap Necrosis and Tissue Perfusion

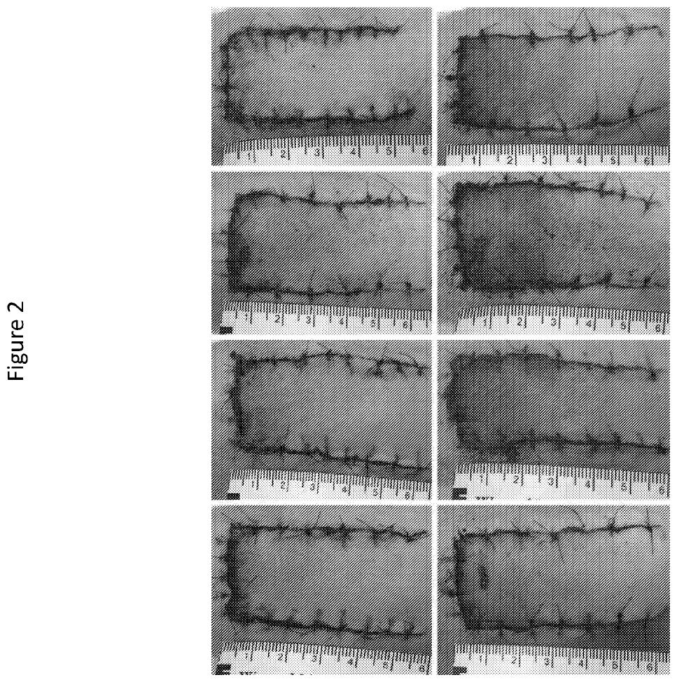

Photographic measurements of necrosis were taken on postoperative days 1, 3, and 7. Flap necrosis was determined grossly by the presence of cyanosis and congestion on postoperative day 1 (FIG. 2) and the presence of scab formation and loss of skin elasticity on subsequent postsurgical days. At 7 days, tissue perfusion was assessed by means of intraperitoneal injection of 1 ml of sodium fluorescein 10% at 60 mg/kg and fluorescent imaging with an in vivo imaging system (IVIS Kinetic; PerkinElmer, Waltham, Mass.) using LIVING IMAGE® software with a 465-nm excitation and green fluorescent protein emission filter. Necrotic area was calculated using ImageJ Software (National Institutes of Health, Bethesda, Md.). Digital photographs and IVIS images were evaluated and analyzed by an investigator blinded to the assigned treatment group.

Histologic Analysis and Immunohistochemistry

Tissues from the proximal and distal parts of the flaps were collected on postoperative day 7 and were fixed in 10% paraformaldehyde and embedded in paraffin, with representative sections depicted in FIG. 1. For histologic evaluation and CD31 immunohistochemical staining, sections taken 6 cm from the proximal flap were deparaffinized in xylene and rehydrated in a series of ethanol washes. For detection of CD31+ cells, sections were incubated with anti-mouse CD31 antibody (BD Pharmingen, San Jose, Calif.) at a dilution of 1:200 at 4° C. overnight. For analysis of angiogenesis, the CD31+ vessels in five fields were counted by light microscopy (20×) for each group. A TdT In Situ Cell Death Detection Kit (R&D Systems, Minneapolis, Minn.) was used to compare apoptotic protein expression in sections taken 4 cm from the proximal flap. The ratio of terminal deoxynucleotidyl transferase-mediated dUDP end-labeling-positive cells was calculated according to the manufacturer's protocol. Terminal deoxynucleotidyl transferase-mediated dUDP end-labeling-positive cells were also counted by light microscopy (20×) in 10 fields.

For analysis of HIF-1α, sections were taken 4 cm from the proximal flap and incubated with anti-rabbit HIF-1α antibody (Novus Biologicals, Littleton, Colo.) at a dilution of 1:1600. A Discovery Ultra immunohistochemical system (Ventana Medical Systems, Oro Valley, Ariz.) was used for staining with OmniMap anti-Rb horseradish peroxidase and red chromogen was used for detection. HIF-1α-positive nuclei in the epidermis were counted by light microscopy (20×) in five fields. All immunohistochemical measurements were performed by a blinded investigator.

Determining Tissue VEGF Concentration

Tissue samples were harvested on postoperative day 7 and dissected on ice, submerged in liquid nitrogen, then stored at −80° C. Tissue samples from tissue sections taken at the same distance 6 cm from the proximal flap were then homogenized in lysis buffer containing 100 mM Tris hydrochloride (pH 7.4), 150 mmol sodium chloride, 1% Triton X-100, 0.5% sodium deoxycholate, and 1 μg/ml protease inhibitor cocktail (Sigma Aldrich, St. Louis, Mo.). The samples were centrifuged to pellet the debris, and the supernatants were analyzed following the manufacturer's protocol for a rat tissue extract VEGF enzyme-linked immunosorbent assay kit (Sigma-Aldrich).

Ethics Statement and Side Effect Monitoring

The animals were treated according to institutional animal care and use committee guidelines. Animals were double-housed for the preoperative period, then single-housed in the immediate postoperative period to protect flap integrity. Isoflurane anesthesia was used for all operations and daily intraperitoneal injections. Elizabethan collars were placed on rats to prevent ingestion of the topical solution after application. No dressing was used over the topical application site due to absorption. Animals were observed daily for side effects, including wound infections or dehiscence. Weights were taken on the day of surgery and on postoperative day 7. Blood samples were obtained on postoperative day 7 immediately before the animals were euthanized by means of cardiac puncture to determine hemoglobin, hematocrit, white blood cell count, and platelet count. Animals were euthanized by means of intracardiac potassium chloride on postoperative day 7, and gross necropsies were performed by a veterinarian to assess macroscopic organ changes after 14 total days of treatment.

Statistical Analysis

Parametric data were expressed as means±SE and compared using the t test or analysis of variance. All tests were two-sided and were considered statistically significant for values of p<0.05. Statistical analysis was performed using JMP Version 13 (SAS Institute, Inc., Cary, N.C.) or GraphPad Prism (Version 7.0a; GraphPad Software, Inc., La Jolla, Calif).

Results Dimethyloxalylglycine Treatment Significantly Enhances Postsurgical Flap Viability

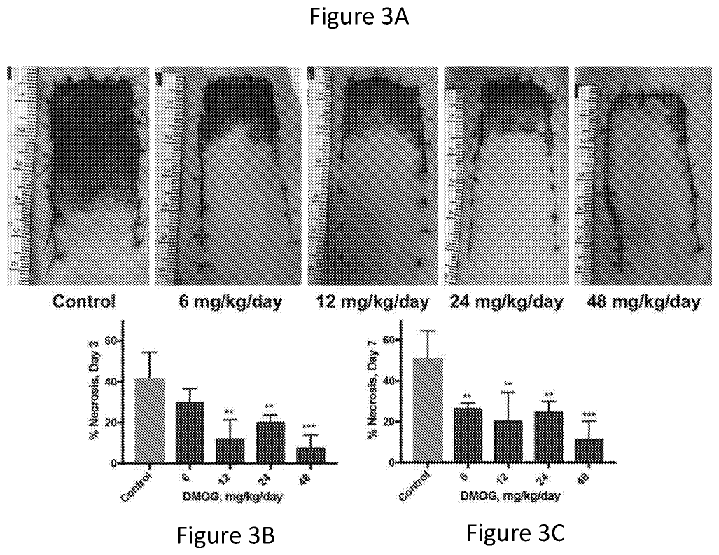

To assess the effect of prolyl hydroxylase inhibition on skin flap necrosis, a 3×6-cm dorsal skin flap model was used to achieve distal flap necrosis. Decreased cyanosis was observed in treated flaps on day 1 (FIG. 2). On postoperative day 3, dimethyloxalylglycine treatment led to significantly reduced mean percentage of skin flap necrosis compared with controls (41.3±3.8 percent) when administered at 12 mg/kg/day (12.3±4.5 percent; p=0.001), 24 mg/kg/day (20.3±1.8 percent; p=0.008), and 48 mg/kg/day (7.7±3.1 percent; p<0.001) (FIG. 3B). No difference in skin flap necrosis at postoperative day 3 was observed in the group treated with 6 mg/kg/day dimethyloxalylglycine (30.2±3.9 percent; p=0.13) (FIG. 3B). By postoperative day 7, dimethyloxalylglycine treatment led to reduced flap necrosis at all doses, including 6 mg/kg/day (26.7±1.3 percent; p=0.004), 12 mg/kg/day (20.6±7.0 percent; p=0.002), 24 mg/kg/day (25.0±2.5 percent; p=0.003), and 48 mg/kg/day (11.6±4.4 percent; p<0.001), compared with control animals (50.9±3.9 percent) (FIGS. 3A and 3C).

Prolyl Hydroxylase Inhibition Enhances Skin Flap Tissue Perfusion

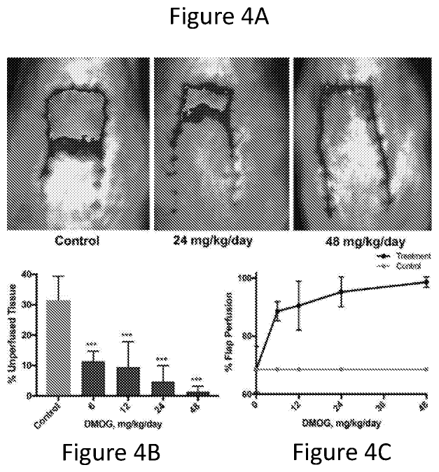

An IVIS kinetics system was used to image skin flap perfusion on postoperative day 7 after sodium fluorescein injection. Dimethyloxalylglycine-treated flaps exhibited a significantly lower percentage of unperfused tissue at postoperative day 7 compared with controls (39.9±3.8 percent) when administered at 6 mg/kg/day (11.4±1.7 percent; p<0.001), 12 mg/kg/day (9.4±4.2 percent; p<0.001), 24 mg/kg/day (4.7±2.6 percent; p<0.001), and 48 mg/kg/day (1.4±0.9 percent; p<0.001) (FIGS. 4A and 4B). Tissue perfusion exhibited a dose-response relationship, with higher dimethyloxalylglycine doses leading to increased tissue perfusion (FIG. 4C).

Topical Dimethyloxalylglycine Application Alone Is Sufficient to Improve Postsurgical Skin Viability

To assess whether topical administration of dimethyloxalylglycine was sufficient to improve skin flap viability, postsurgical skin flap necrosis was compared in animals treated with either topical dimethyloxalylglycine (n=4) or intraperitoneal dimethyloxalylglycine (n=4) at 48 mg/kg/day.

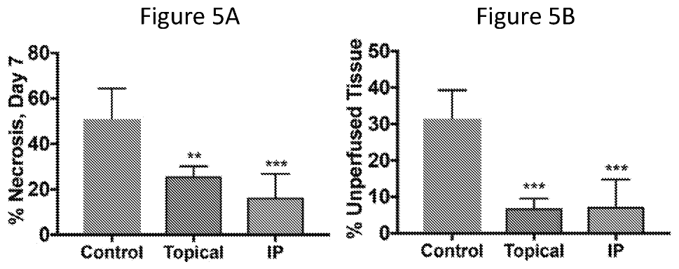

On postsurgical day 3, compared to controls (41.3±3.8 percent), a significant reduction in postsurgical flap necrosis percentage was observed in animals treated with both topical dimethyloxalylglycine (18.9±2.8 percent; p=0.005) and intraperitoneal dimethyloxalylglycine alone (14.7±2.6 percent; p=0.002). Similarly, on postsurgical day 7, reduced percentage of flap necrosis was observed in animals treated with topical dimethyloxalylglycine (25.7±2.3 percent; p=0.003) and intraperitoneal dimethyloxalylglycine alone (16.3±5.2 percent; p<0.001) compared with controls (50.9±3.9 percent) (FIG. 5A). Finally, dimethyloxalylglycine-treated flaps exhibited a significantly higher percentage of overall flap perfusion at postoperative day 7 compared with controls (31.4±2.3 percent) when administered both topically (6.9±1.3 percent; p<0.001) and intraperitoneally alone (7.2±3.8; p<0.001) (FIG. 5B).

Topical Prolyl Hydroxylase Inhibition Increases Nuclear HIF-1α in the Epidermis

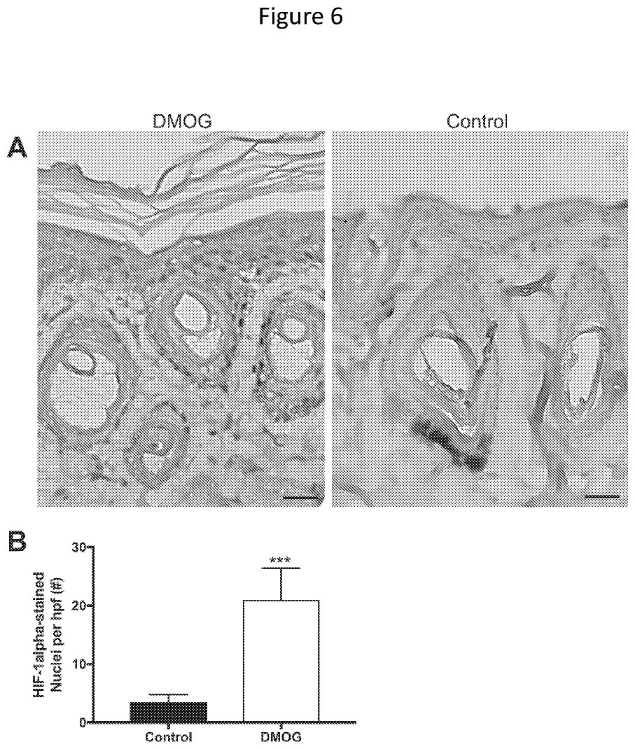

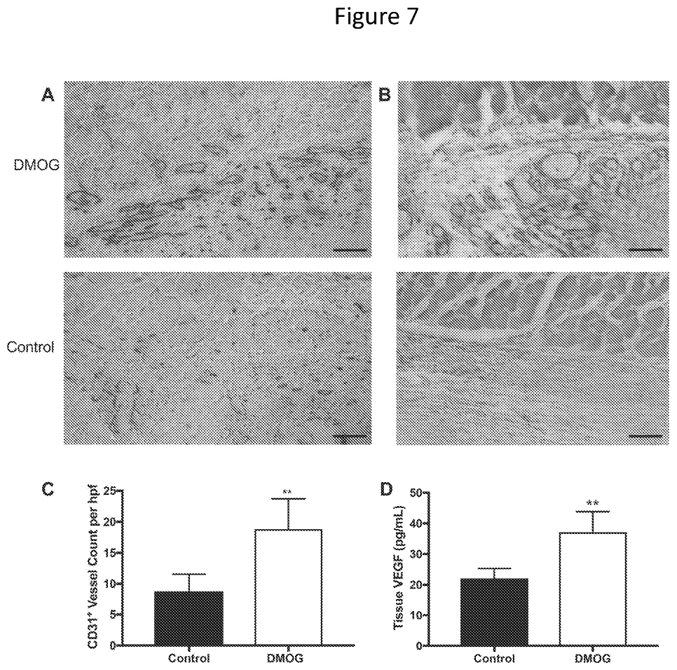

Given that prolyl hydroxylase promotes degradation of HIF-1α, it was examined whether application of prolyl hydroxylase inhibitors increased epidermal HIF-1α. In skin flaps harvested 4 cm from the proximal flap from treated and untreated animals, dimethyloxalylglycine-treated skin flaps exhibited a significantly greater epidermal HIF-1α staining compared with controls, with the number of HIF-1α-stained nuclei per high-power field significantly higher in dimethyloxalylglycine-treated skin flaps compared with controls (21.0±2.7 versus 3.5±0.6; p<0.001) (see FIGS. 6 and 7).

Prolyl Hydroxylase Inhibition Promotes Angiogenesis through VEGF Transcription

Given the proangiogenic mechanism of prolyl hydroxylase inhibitors through VEGF upregulation and considering that necrosis in the McFarlane model stems from failure of the blood supply through pedicle disruption, neovascularization in the proximal skin flap was examined after 14 total days of treatment. In skin flaps harvested proximal to the pedicle at the same distance for treated and untreated animals, dimethyloxalylglycine-treated animals exhibited greater neovascularization on hematoxylin and eosin-stained tissue (see FIG. 7). The number of CD31-labeled vessels harvested from the skin flap proximal to the pedicle was significantly higher in dimethyloxalylglycine-treated skin flaps compared with controls (18.8±2.2 versus 8.8±1.2; p=0.004). Similarly, tissue VEGF concentrations measured with enzyme-linked immunosorbent assay were significantly higher in the dimethyloxalylglycine-treated skin flaps (37.1±3.4 pg/ml versus 22.0±1.7 pg/ml; p=0.007).

Dimethyloxalylglycine Treatment Suppresses Expression of Apoptotic Proteins

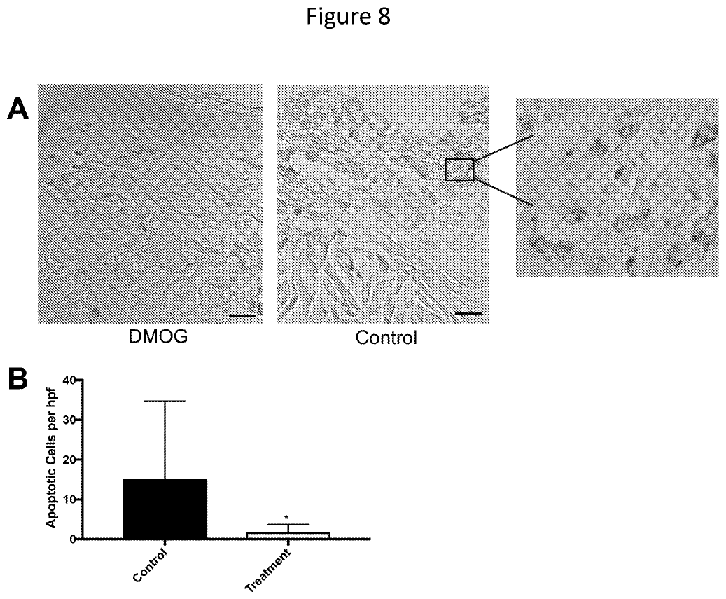

Because HIF-1α has been linked to reduced expression of apoptotic proteins, and portions of the ischemic flap proximal to necrotic areas must resist apoptosis to survive ischemic insult, terminal deoxynucleotidyl transferase-mediated dUDP end-labeling staining was used to examine apoptotic protein expression. Dimethyloxalylglycine-treated sections had reduced numbers of apoptotic cells per high-power field at 20× magnification compared with controls (1.7±0.6 versus 15.1±6.2; p=0.045) (see FIG. 8).

Dimethyloxalylglycine Treatment Does Not Lead to Polycythemia or Gross Systemic Toxicity in Rats

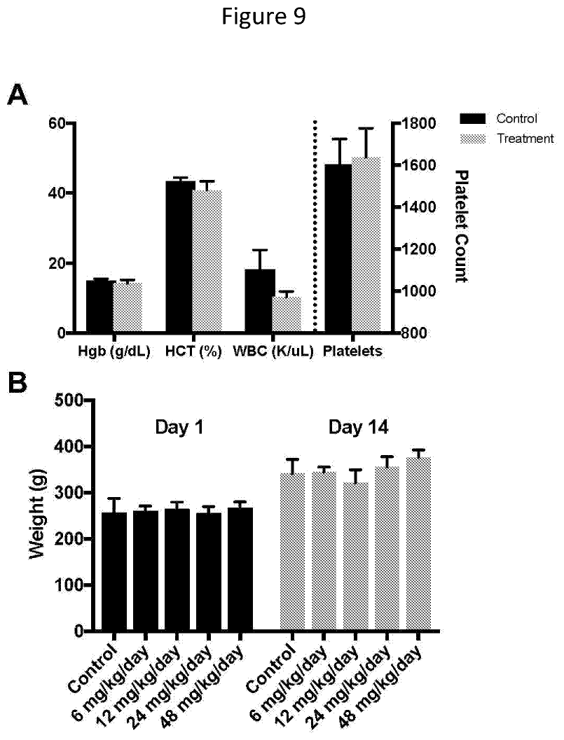

There were no significant differences in mean hemoglobin (dimethyloxalylglycine, 14.2±1.0 g/dl; control, 15.1±0.5 g/dl; p=0.3) or hematocrit (dimethyloxalylglycine, 40.8±2.6 percent; control, 43.3±1.1 percent; p=0.2) between the groups. In addition, there were no significant differences in mean white blood cell count (dimethyloxalylglycine, 10.4±1.6×10³/p1; control, 18.2±5.6×10³/p1; p=0.08) and platelet count (dimethyloxalylglycine, 1510±247×10⁹/liter; control, 1446±218×10⁹/liter; p=0.8) (see FIG. 9).

No wound infections, dehiscence, or behavioral changes in feeding or activity were observed throughout the experimental time course. There were no differences in preoperative weights taken on experimental day 1 or day 14 between control animals and treated animals at all doses. Gross necropsy specimens obtained after 14 total days of treatment showed no evidence of cardiovascular changes, splenomegaly, or polycythemia.

### DISCUSSION

This example demonstrates that tissue preconditioning with dimethyloxalylglycine leads to significantly enhanced flap viability, as evidenced by both reduction in gross flap necrosis and increased tissue perfusion. Prolyl hydroxylase inhibition increases HIF-1α expression, promoting VEGF transcription and downstream neovascularization. The example also shows that dimethyloxalylglycine preconditioning appears to lack obvious systemic toxicity related to polycythemia or wound healing, highlighting the potential utility of prolyl hydroxylase inhibitors as novel agents to improve tissue adaptation to ischemia.

Because of significant morbidity and health care expenditures associated with postoperative tissue necrosis, identifying pharmacologic agents that precondition skin to better withstand ischemic insult is an area of active investigation. To date, the majority of this research has focused on agents targeting neovascularization, including vasodilators, VEGF delivery vehicles, sildenafil, and minoxidil. Mechanical forces that promote flap perfusion through conditional hypoxia, such as external suction, local heat treatment, and electric stimulation, have also been investigated as agents of preconditioning. Other research has attempted to capitalize on cellular adaptation to oxidative stress by using antioxidants, including N-acetylcysteine, melatonin, and calcitriol, to reduce flap necrosis. Together, these strategies have expanded knowledge of the mechanism governing flap necrosis and tissue adaptation to ischemia. However, the marginality of reported improvements precluded larger animal studies, clinical trials, and clinical application of these approaches. Thus, the need for novel pharmacologic approaches that improve the viability of ischemic tissues and that are clinically applicable remains.

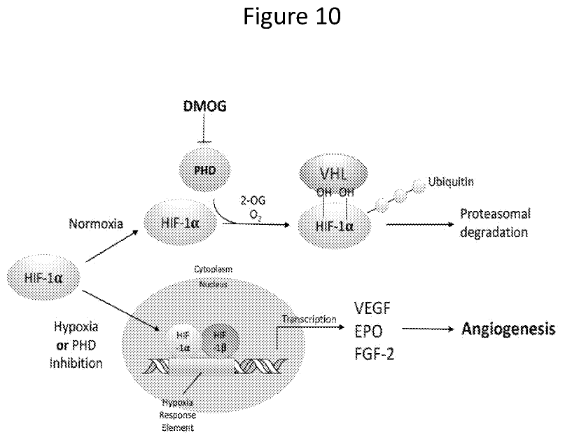

Preconditioning agents with mechanisms initiating metabolic adaptation to hypoxia could represent a more effective approach to improve flap viability. The HIF pathway is considered the master switch of tissue adaptation to hypoxic environments, and activity of HIF-la is counterbalanced by prolyl hydroxylase enzymes, as shown in FIG. 10. Under hypoxic conditions, HIF-1α activation promotes transcription of proangiogenic genes, including VEGF. Prolyl hydroxylase enzymes function to inhibit HIF-1α under normoxic conditions; thus, pharmacologic inhibition of prolyl hydroxylase recapitulates the HIF-1 hypoxia-response sequence, enabling HIF-1α transcription regulation and downstream angiogenesis. Currently, preclinical trials of prolyl hydroxylase inhibitors as agents to mitigate ischemia-reperfusion injury in vascular grafts, kidney transplantation, and myocardial infarctions are underway. Furthermore, because of the downstream effects of prolyl hydroxylase inhibition on transcription of erythropoietin, phase II and III clinical trials testing prolyl hydroxylase inhibitors including Roduxastat (AstraZeneca, Cambridge, United Kingdom), Molidastat (Bayer, Leverkusen, Germany), Daprodustat (GlaxoSmithKline, Brentford, United Kingdom), and Vadadustat (Akebia Therapeutics, Inc., Cambridge, Mass.) as novel agents to treat chronic kidney disease-induced anemia are ongoing. These clinical trials have demonstrated promising results and, thus far, few adverse side effects have been reported, even with daily administration.

Described herein is a novel application of prolyl hydroxylase inhibitors to precondition tissue before flap elevation. Indeed, the present disclosure demonstrates the utility of both systemic and topical prolyl hydroxylase inhibitors for improving the viability of ischemic tissue. A nearly threefold increase in flap viability was observed after topical application and intraperitoneal administration of dimethyloxalylglycine, an improvement that far exceeds the findings of previous studies using similar animal models. The significant effect of dimethyloxalylglycine on flap viability likely stems from prolyl hydroxylase inhibitors acting early in the HIF pathway, thereby stimulating multiple downstream targets, including cellular adaptation to hypoxia, neovascularization, and apoptosis, as opposed to targeting one specific effect as in previous pharmacologic approaches. 14 days of dimethyloxalylglycine administration was sufficient to both induce angiogenesis and reduce apoptosis, evidenced by increased numbers of CD31-stained vessels and reduced numbers of terminal deoxynucleotidyl transferase-mediated dUDP end-labeling-stained apoptotic cells, respectively, in treated animals.

Furthermore, the endpoint of the mechanism of prolyl hydroxylase inhibitors was verified, showing that prolyl hydroxylase inhibition increases HIF-1α expression and downstream VEGF transcription. The clinical applicability of this therapeutic approach would be especially relevant to patients with preexisting impaired wound healing caused by diabetes, obesity, and malnutrition, and in patients with previously irradiated skin. Given the relative ease of clinically translating this approach into a short course of topical or oral drugs before flap surgery and the significant improvement in flap viability demonstrated in this study, prolyl hydroxylase inhibitors are a promising novel solution to tissue ischemia and flap necrosis.

Because of the pharmacologic mechanism, dose-dependent increase in hemoglobin is a potential side effect of prolyl hydroxylase inhibitors when used for applications outside of chronic kidney disease-induced anemia. Although reported side effects have been minimal, hypertension resulting from polycythemia has been cited as the most common adverse effect in clinical trials of Molidastat and Vadadustat. However, at the topical and systemic doses used in the studies described herein, no increase in hemoglobin occurred in dimethyloxalylglycine-treated animals. Another potential feared side effect of prolyl hydroxylase inhibitors is stimulation of tumorigenesis, as HIF-1α has been reported to be overexpressed in tumors. However, in animal studies of mice with disseminated metastases treated with prolyl hydroxylase inhibitors, no growth of existing tumors, increased tumor angiogenesis, or increased metastatic potential occurred after treatment, suggesting that the mechanism of prolyl hydroxylase inhibitors may home to severely ischemic tissues. Furthermore, no tumorigenic effects have been reported in ongoing clinical trials. In the study described herein, no macroscopic organ changes were observed on gross necropsy suggestive of tumorigenesis or polycythemia in dimethyloxalylglycine-treated animals. Based on these observations, the dose of prolyl hydroxylase inhibitors necessary to achieve reductions in flap necrosis may not have significant associated systemic side effects in rodents, and the homing of prolyl hydroxylase inhibitor effect to severely ischemic tissues may reduce the adverse effects of this approach in normal skin. However, larger animal models are necessary to further evaluate the safety profile and efficacy of prolyl hydroxylase inhibitors for this application.

The results of this example demonstrate that dimethyloxalylglycine leads to a clear and significant reduction in postoperative flap necrosis.

### REFERENCES

1. Kerrigan C L. Skin flap failure: Pathophysiology. Plast Reconstr Surg. 1983; 72:766-777.
2. Harder Y, Amon M, Laschke M W, et al. An old dream revitalised: Preconditioning strategies to protect surgical flaps from critical ischaemia and ischaemia-reperfusion injury. J Plast Reconstr Aesthet Surg. 2008; 61:503-511.
3. Finseth F, Adelberg M G. Prevention of skin flap necrosis by a course of treatment with vasodilator drugs. Plast Reconstr Surg. 1978; 61:738-743.
4. Seify H, Bilkay U, Jones G. Improvement of TRAM flap viability using human VEGF-induced angiogenesis: A comparative study of delay techniques. Plast Reconstr Surg. 2003; 112:1032-1039.
5. Michlits W, Mittermayr R, Schafer R, Redl H, Aharinejad S. Fibrin-embedded administration of VEGF plasmid enhances skin flap survival. Wound Repair Regen. 2007; 15:360-367.
6. Tetik Menevse G, Islamoglu K, Ege Ozgentas H. Expansion of surviving skin paddle of neurocutaneous island flaps in rats by VEGF. J Reconstr Microsurg. 2007; 23:99-105.
7. Gersch R P, Fourman M S, Phillips B T, et al. AdVEGF-A116A+ preconditioning of murine ischemic skin flaps is comparable to surgical delay. Plast Reconstr Surg Glob Open 2015; 3:e494.
8. Gumu N, Odemi Y, Tuncer E, Yilmaz S. The effect of topical minoxidil pretreatment on nonsurgical delay of rat cutaneous flaps: Further studies. Aesthetic Plast Surg. 2013; 37:809-815.
9. Gozu A, Poda M, Takin E I, et al. Pretreatment with octreotide modulates iNOS gene expression, mimics surgical delay, and improves flap survival. Ann Plast Surg. 2010; 65:245-249.
10. Jonsson K, Hunt T K, Brennan S S, Mathes S J. Tissue oxygen measurements in delayed skin flaps: A reconsideration of the mechanisms of the delay phenomenon. Plast Reconstr Surg. 1988; 82:328-336.
11. Semenza G L. Hypoxia-inducible factors in physiology and medicine. Cell 2012; 148:399-408
12. Heim C, Bernhardt W, Jalilova S, et al. Prolyl-hydroxylase inhibitor activating hypoxia-inducible transcription factors reduce levels of transplant arteriosclerosis in a murine aortic allograft model. Interact Cardiovasc Thorac Surg. 2016; 22:561-570.
13. Bernhardt W M, Gottmann U, Doyon F, et al. Donor treatment with a PHD-inhibitor activating HIFs prevents graft injury and prolongs survival in an allogenic kidney transplant model. Proc Natl Acad Sci USA 2009; 106:21276-21281.
14. Rahtu-Korpela L, Maatta J, Dimova E Y, et al. Hypoxiainducible factor prolyl 4-hydroxylase-2 inhibition protects against development of atherosclerosis. Arterioscler Thromb Vasc Biol. 2016; 36:608-617.
15. Reischl S, Li L, Walkinshaw G, Flippin L A, Marti H H, Kunze R. Inhibition of HIF prolyl-4-hydroxylases by FG-4497 reduces brain tissue injury and edema formation during ischemic stroke. PLoS One 2014; 9:e84767.
16. Karuppagounder S S, Alim I, Khim S J, et al. Therapeutic targeting of oxygen-sensing prolyl hydroxylases abrogates ATF4-dependent neuronal death and improves outcomes after brain hemorrhage in several rodent models. Sci Transl Med. 2016; 8:328ra29.
17. Maxwell P H, Eckardt K U. HIF prolyl hydroxylase inhibitors for the treatment of renal anaemia and beyond. Nat Rev Nephrol. 2016; 12:157-168.
18. Gupta N, Wish J B. Hypoxia-inducible factor prolyl hydroxylase inhibitors: A potential new treatment for anemia in patients with CKD. Am J Kidney Dis. 2017; 69:815-826. 19. Carney E F. Therapy: PHD inhibitors correct anaemia in CKD. Nat Rev Nephrol. 2016; 12:3.
20. Provenzano R, Besarab A, Sun C H, et al. Oral hypoxia-inducible factor prolyl hydroxylase inhibitor Roxadustat (FG-4592) for the treatment of anemia in patients with CKD. Clin J Am Soc Nephrol. 2016; 11:982-991.
21. Besarab A, Provenzano R, Hertel J, et al. Randomized placebo-controlled dose-ranging and pharmacodynamics study of roxadustat (FG-4592) to treat anemia in nondialysisdependent chronic kidney disease (NDD-CKD) patients. Nephrol Dial Transplant. 2015; 30:1665-1673.
22. Yuan Q, Bleiziffer O, Boos A M, et al. PHDs inhibitor DMOG promotes the vascularization process in the AV loop by HIF-1α up-regulation and the preliminary discussion on its kinetics in rat. BMC Biotechnol. 2014; 14:112.
23. Marchbank T, Mahmood A, Harten S, Maxwell P H, Playford R J. Dimethyloxalyglycine stimulates the early stages of gastrointestinal repair processes through VEGF-dependent mechanisms. Lab Invest. 2011; 91:1684-1694.
24. Poynter J A, Manukyan M C, Wang Y, et al. Systemic pretreatment with dimethyloxalylglycine increases myocardial HIF-1α and VEGF production and improves functional recovery after acute ischemia/reperfusion. Surgery 2011; 150:278-283.
25. Dallatu M K, Nwokocha E, Agu N, et al. The role of hypoxiainducible factor/prolyl hydroxylation pathway in deoxycorticosterone acetate/salt hypertension in the rat. J Hypertens (Los Angel.) 2014; 3:184.
26. Duscher D, Januszyk M, Maan Z N, et al. Comparison of the hydroxylase inhibitor dimethyloxalylglycine and the iron chelator deferoxamine in diabetic and aged wound healing. Plast Reconstr Surg. 2017; 139:695e-706e.
27. McFarlane R M, DeYoung G, Henry R A. The design of a pedicle flap in the rat to study necrosis and its prevention. Plast Reconstr Surg. 1965; 35:177-182.
28. Choi J A, Lee K C, Kim M S, Kim S K. Comparison of prostaglandin E1 and sildenafil citrate administration on skin flap survival in rats. Arch Craniofac Surg. 2015; 16:73-79.
29. Giatsidis G, Cheng L, Haddad A, et al. Noninvasive induction of angiogenesis in tissues by external suction: Sequential optimization for use in reconstructive surgery. Angiogenesis 2018; 21:61-78.
30. Mehta S, Rolph R, Cornelius V, Harder Y, Farhadi J. Local heat preconditioning in skin sparing mastectomy: A pilot study. J Plast Reconstr Aesthet Surg. 2013; 66:1676-1682.
31. Dogan F, Ozyazgan I. Flap preconditioning by electrical stimulation as an alternative to surgical delay: Experimental study. Ann Plast Surg. 2015; 75:560-564.
32. Tunc S, Kesiktas E, Yilmaz Y, et al. Assessing the effects of melatonin and N-acetylcysteine on the McFarlane flap using a rat model. Plast Surg (Oakv.) 2016; 24:204-208.
33. Zhou K L, Zhang Y H, Lin D S, Tao X Y, Xu H Z. Effects of calcitriol on random skin flap survival in rats. Sci Rep. 2016; 6:18945.
34. Rabinowitz M H. Inhibition of hypoxia-inducible factor prolyl hydroxylase domain oxygen sensors: Tricking the body into mounting orchestrated survival and repair responses. J Med Chem. 2013; 56:9369-9402.
35. Barnucz E, Veres G, Hegedus P, et al. Prolyl-hydroxylase inhibition preserves endothelial cell function in a rat model of vascular ischemia reperfusion injury. J Pharmacol Exp Ther. 2013; 345:25-31.
36. Xie L, Pi X, Wang Z, He J, Willis M S, Patterson C. Depletion of PHD3 protects heart from ischemia/reperfusion injury by inhibiting cardiomyocyte apoptosis. J Mol Cell Cardiol. 2015; 80:156-165.
37. Zimmermann A S, Morrison S D, Hu M S, et al. Epidermal or dermal specific knockout of PHD-2 enhances wound healing and minimizes ischemic injury. PLoS One 2014; 9:e93373.
38. Takaku M, Tomita S, Kurobe H, et al. Systemic preconditioning by a prolyl hydroxylase inhibitor promotes prevention of skin flap necrosis via HIF-1-induced bone marrow-derived cells. PLoS One 2012; 7:e42964.
39. Rankin E B, Giaccia A J. The role of hypoxia-inducible factors in tumorigenesis. Cell Death Differ. 2008; 15:678-685.
40. Harnoss J M, Platzer L K, Burhenne J, et al. Prolyl hydroxylase inhibition enhances liver regeneration without induction of tumor growth. Ann Surg. 2017; 265:782-791.
41. Martin E R, Smith M T, Maroni B J, Zuraw Q C, deGoma E M. Clinical trial of Vadadustat in patients with anemia secondary to stage 3 or 4 chronic kidney disease. Am J Nephrol. 2017; 45:380-388.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of reducing ischemic damage to a surgical incision in a tissue of subject, which comprises contacting said surgical incision topically with an effective dose of a HIF-1α potentiating agent, thereby reducing ischemic damage to the surgical incision.

2-4. (canceled)

5. The method of claim 1, wherein the HIF-1α potentiating agent transdermally penetrates the tissue.

6. The method of claim 1, wherein the HIF-1α potentiating agent upregulates expression of HIF-1α.

7. The method of claim 1, wherein the HIF-1α potentiating agent inhibits the activity of prolyl hydroxylase (PHD), wherein the HIF-1α potentiating agent comprises dimethyloxalylglycine (DMOG).

8. (canceled)

9. The method of claim 1, wherein the HIF-1α potentiating agent is provided in a lotion or gel.

10-19. (canceled)

20. A method of enhancing tissue viability and vascularity following an ischemic insult in a subject, which comprises contacting said tissue topically with an effective dose of a HIF-1α potentiating agent, thereby enhancing tissue viability and vascularity following the ischemic insult.

21. The method of claim 20, wherein the ischemic insult is a surgical incision.

22. The method of claim 20, wherein the HIF-1α potentiating agent transdermally penetrates the tissue.

23. The method of claim 20, wherein the HIF-1α potentiating agent upregulates expression of HIF-1α.

24. The method of claim 20, wherein the HIF-1α potentiating agent inhibits the activity of prolyl hydroxylase (PHD), wherein the HIF-1α potentiating agent comprises dimethyloxalylglycine (DMOG).

25. The method of claim 20, wherein the HIF-1α potentiating agent is provided in a lotion or gel.

26. A method for preconditioning tissue to resist an ischemic insult, which comprises contacting said tissue topically with an effective dose of a HIF-1α potentiating agent prior to the ischemic insult.

27. The method of claim 26, wherein the ischemic insult is a surgical incision.

28. The method of claim 26, wherein the HIF-1α potentiating agent transdermally penetrates the tissue.

29. The method of claim 26, wherein the HIF-1α potentiating agent upregulates expression of HIF-1α.

30. The method of claim 26, wherein the HIF-1α potentiating agent inhibits the activity of prolyl hydroxylase (PHD), wherein the HIF-1α potentiating agent comprises dimethyloxalylglycine (DMOG).

31. The method of claim 26, wherein the HIF-1α potentiating agent is provided in a lotion or gel.

32. The method of claim 26, wherein the HIF-1α potentiating agent is administered 1-10 hours prior to the ischemic insult.

33. The method of claim 26,
wherein the HIF-1α potentiating agent is administered 1-7 days prior to the ischemic insult, and/or
wherein the HIF-1α potentiating agent is administered 1-7 days after the ischemic insult.

34. The method of claim 26, which further comprises administering the HIF-1α potentiating agent after the ischemic insult.
